(19) United States
(12) Patent Application Publication
(10) Pub. No.: US 2024/0325774 A1
(43) Pub. Date: Oct. 3, 2024

(54) Methods of Treating Metabolic Disorders with an Implanted Device

(71) Applicant: Boston Scientific Scimed, Inc., Maple Grove, MN (US)

(72) Inventors: Andrew Brian Graveley, Shoreview, MN (US); Daniel J. Foster, Lino Lakes, MN (US); Kyle True, Minneapolis, MN (US); Brian L. Schmidt, White Bear Lake, MN (US); Devon N. Arnholt, Shoreview, MN (US)

(73) Assignee: Boston Scientific Scimed, Inc., Maple Grove, MN (US)

(21) Appl. No.: 18/619,951

(22) Filed: Mar. 28, 2024

Related U.S. Application Data
(60) Provisional application No. 63/455,085, filed on Mar. 28, 2023.

(51) Int. Cl. A61N 2/06; A61N 2/00; A61N 2/02

(52) U.S. Cl. CPC A61N 2/06; A61N 2/006; A61N 2/02

(57) ABSTRACT

Metabolic disorders such as Type 2 diabetes may be treated using a system that is adapted to modulate an hepatic neural plexus of a patient. The system includes a stimulation device adapted for implantation near the patient's hepatic neural plexus and to provide mechanical energy to the patient's hepatic neural plexus, a source of excitation energy adapted to provide excitation energy to the stimulation device, and a controller that is operably coupled to the source of excitation energy and is adapted to control the source of excitation energy.

### CROSS-REFERENCE TO RELATED APPLICATIONS

This application claims the benefit of priority under 35 U.S.C. § 119 of U.S. Provisional Application No. 63/455,085, filed Mar. 28, 2023, the entire disclosure of which is hereby incorporated by reference.

### TECHNICAL FIELD

The disclosure relates generally to medical devices and more particularly to implantable medical devices that are adapted for use in treating metabolic disorders.

### BACKGROUND

Patients may suffer from any of a variety of different metabolic disorders. An example is Type 2 diabetes, which is a condition in which a patient's pancreas does not necessarily produce sufficient insulin to accommodate the patient's intake of carbohydrates. This is distinguished from Type I diabetes, which is a condition in which the patient's pancreas is no longer producing any insulin. Type I diabetes is treated using injected insulin. In many cases, Type 2 diabetes is managed with diet and weight loss. It will be appreciated that some patients do better in managing their Type 2 diabetes than other patients. Poor management of Type 2 diabetes can lead to a variety of long-term health problems, including atherosclerosis, neuropathy, kidney disease and eye damage, among other possible complications. A need remains for improved techniques, methods and devices for treating metabolic disorders such as Type 2 diabetes.

### SUMMARY

This disclosure provides design, material, manufacturing method, and use alternatives for medical devices. An example may be found in an implantable medical device adapted to modulate an hepatic neural plexus of a patient. The implantable medical device includes a stimulation device adapted to provide energy to the patient's hepatic neural plexus, a power supply adapted to supply power to the stimulation device, and a controller operably coupled to the stimulation device and the power supply and adapted to control operation of the stimulation device.

Alternatively or additionally, the implantable medical device may further include a receiver operably coupled with the controller, the receiver adapted to receive one or more control parameters for controlling the stimulation device.

Alternatively or additionally, the implantable medical device may further include a transmitter, at least one of the receiver and the transmitter being adapted to communicate with an external device.

Alternatively or additionally, at least one of the receiver and the transmitter may be adapted to communicate with an implanted device remote from the implantable medical device.

Alternatively or additionally, the implantable medical device may further include a housing adapted to house the controller and the power supply.

Alternatively or additionally, the housing may be adapted to house the stimulation device.

Alternatively or additionally, the housing may be adapted to be implanted near the hepatic neural plexus of the patient.

Alternatively or additionally, the housing may be adapted to be implanted within an hepatic portal vein of the patient.

Alternatively or additionally, the housing may be adapted to be implanted on or within a heptatoduodenal ligament of the patient.

Alternatively or additionally, the stimulation device may include a pair of electrical leads.

Alternatively or additionally, the stimulation device may include a transducer.

Alternatively or additionally, the transducer may be adapted to use mechanical energy in modulating the hepatic neural plexus of the patient.

Alternatively or additionally, the transducer may be adapted to use electrical energy in modulating the hepatic neural plexus of the patient.

Alternatively or additionally, the transducer may be adapted to use thermal energy in modulating the hepatic neural plexus of the patient.

Alternatively or additionally, the transducer may be adapted to use ultrasonic energy in modulating the hepatic neural plexus of the patient.

Another example may be found in an implantable medical device adapted to modulate an hepatic neural plexus of a patient. The implantable medical device includes a transducer adapted to provide energy to the patient's hepatic neural plexus, a power supply adapted to supply power to the transducer, a controller operably coupled to the transducer and the power supply, the controller adapted to control operation of the transducer, and a receiver operably coupled to the controller, the receiver adapted to receive control parameters for controlling operation of the transducer.

Alternatively or additionally, the control parameters may include one or more of power level, pulse frequency, pulse duration, pulse repetition frequency and duration of time that pulses may be repeated.

Alternatively or additionally, the implantable medical device may further include a sensor that is operably coupled with the controller in order to provide feedback to the controller.

Alternatively or additionally, the implantable medical device may further include a housing that is adapted to be implanted near the hepatic neural plexus of the patient.

Alternatively or additionally, the transducer may include one or more ultrasound transducers.

Another example may be found in an implantable device adapted to stimulate an hepatic neural plexus of a patient in order to modulate a metabolic disorder. The implantable device includes a transducer adapted to provide energy to the patient's hepatic neural plexus, a power supply adapted to supply power to the transducer, a controller operably coupled to the transducer and the power supply, the controller adapted to control operation of the transducer, and a receiver operably coupled to the controller, the receiver adapted to receive control parameters for controlling operation of the transducer.

Alternatively or additionally, the transducer may include one or more ultrasound transducers.

Another example may be found in a system adapted to modulate an hepatic neural plexus of a patient, the system including a stimulation device adapted for implantation near the patient's hepatic neural plexus and to provide mechanical energy to the patient's hepatic neural plexus, a source of excitation energy adapted to provide excitation energy to the stimulation device, and a controller operably coupled to the source of excitation energy and adapted to control the source of excitation energy.

Alternatively or additionally, the stimulation device may be adapted to provide mechanical energy to the patient's hepatic neural plexus by undergoing physical movement.

Alternatively or additionally, the stimulation device may include a permanent magnet and the source of excitation energy may include an inductive coil placed against the patient's skin in order to provide excitation energy to the permanent magnet.

Alternatively or additionally, the permanent magnet may include a neodymium magnet.

Alternatively or additionally, the permanent magnet may include a hermetically encapsulated permanent magnet.

Alternatively or additionally, the permanent magnet may be hermetically encapsulated within a housing.

Alternatively or additionally, the housing may be adapted to be laparoscopically implanted.

Alternatively or additionally, the housing may be adapted to be intravascularly implanted.

Alternatively or additionally, the housing may be adapted to be implanted near the hepatic neural plexus of the patient.

Alternatively or additionally, the housing may be adapted to be implanted within an hepatic portal vein of the patient.

Alternatively or additionally, the housing may be adapted to be implanted on or within a heptatoduodenal ligament of the patient.

Alternatively or additionally, the controller may be adapted to provide an alternating current to the inductive coil in order to generate an alternating magnetic field that provides the excitation energy to the stimulation device.

Alternatively or additionally, the controller may be adapted to provide an alternating current to the inductive coil at an AC frequency ranging from 30 Hertz (Hz) to 20,000 Hz.

Alternatively or additionally, the stimulation device may include a piezoelectric material and the source of excitation energy may include a source of electricity.

Alternatively or additionally, the stimulation device may include an eccentric rotating mass.

Another example may be found in a system adapted to modulate an hepatic neural plexus of a patient, system including a magnetic stimulation device adapted for implantation near the patient's hepatic neural plexus and to provide mechanical energy to the patient's hepatic neural plexus, and an external magnetic field generator adapted to excite the magnetic stimulation device and cause movement of the magnetic stimulation device.

Alternatively or additionally, the magnetic stimulation device may include a neodymium magnet.

Alternatively or additionally, the magnetic stimulation device may include a hermetically encapsulated permanent magnet.

Alternatively or additionally, the external magnetic field generator may be adapted to generate an alternating magnetic field in response to an applied AC current.

Another example may be found in a system adapted to modulate an hepatic neural plexus of a patient, the system including an encapsulated permanent magnet adapted for implantation near the patient's hepatic neural plexus and to provide mechanical energy to the patient's hepatic neural plexus, and an external magnetic field generator adapted to generate an alternating magnetic field. The encapsulated permanent magnet oscillates position in response to the alternating magnetic field.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic block diagram of an illustrative implantable medical device that is adapted to modulate a hepatic neural plexus of a patient;

FIG. 2 is a schematic view of an anatomy showing implantation of an illustrative implantable medical device;

FIG. 3 is a schematic view of a portion of an illustrative implantable medical device;

FIG. 4 is a schematic view of a portion of an illustrative implantable medical device;

FIG. 5 is a schematic view of an anatomy showing delivery routes for implanting an implantable medical device;

FIG. 6 is a schematic view of an anatomy showing implantation of an illustrative implantable medical device;

FIG. 7 is a schematic view of an illustrative housing for an implantable medical device;

FIG. 8 is a schematic view of an illustrative intraluminal lead;

FIG. 9 is a schematic view of an illustrative interstitial lead;

FIG. 10 is a schematic view of an illustrative lead having a J-shaped distal region;

FIG. 11 is a schematic view of an illustrative tube-like stent;

FIG. 12 is a schematic view of an illustrative cuff lead design;

FIG. 13 is a schematic view of an anatomy showing an illustrative medical system including an implantable medical device implanted intravascularly and a corresponding external coil; and

FIG. 14 is a schematic view of an anatomy showing an illustrative medical system including an implantable medical device implanted laparoscopically and a corresponding external coil.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Many patients suffer from a variety of different metabolic disorders. Type 2 diabetes is an example of a metabolic disorder that impacts a large number of patients. If not treated adequately, Type 2 diabetes can lead to a number of health problems including but not limited to atherosclerosis, neuropathy, kidney disease, and eye damage. In severe cases, dialysis, kidney transplants, or even amputation of the lower limbs may be required. Despite these possibly severe consequences, many patients have poor compliance to the lifestyle changes (including diet and exercise), medications and frequent testing that are important in treating Type 2 diabetes.

It has been determined that in some cases, stimulation of the hepatic neural plexus may improve glucose homeostasis. The hepatic neural plexus is a network of interwoven nerves that are located in the porta hepatis region near the liver. Stimulation of the hepatic neural plexus for as little as three minutes a day may help patients who have Type 2 diabetes. While it may be possible to provide such stimulation from a position exterior to the patient, it will be appreciated that there are potential difficulties with this. For example, many patients who have Type 2 diabetes also suffer from obesity. Excess abdominal fat may interfere with external stimulation, for example, by requiring higher stimulation power levels. External stimulation may also lead to possible problems with correctly aiming the stimulation, for example.

In some instances, the hepatic neural plexus may be stimulated from a position within the patient. An implantable medical device may be adapted to be implanted at a position at or near the hepatic neural plexus, for example, and may be adapted to appropriately stimulate the hepatic neural plexus from a close-by position within the patient. It will be appreciated that by implanting such a device, the device will be closer to the nerve or nerves being stimulated, and thus less energy may be needed. In some instances, an implanted stimulation device does not require imaging to fine tune aiming with each use. An implanted stimulation device does not require modification to account for variations in BMI (body mass index) between patients. In some instances, an implanted stimulation device may utilize mechanical energy, electric, or thermal energy to modulate the hepatic neural plexus to metabolic disorders including diabetes or obesity. While the disclosure is directed to impacting the hepatic neural plexus for treating metabolic disorders such as diabetes, it will be appreciated that the implantable stimulation devices described herein may be used to stimulate any of a variety of different nerves or nerve bundles in order to affect any of a variety of metabolic disorders.

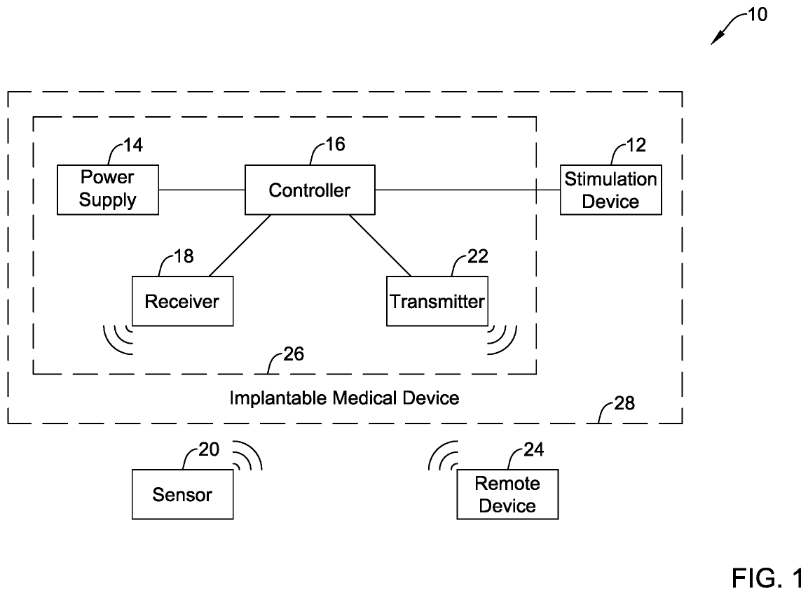

FIG. 1 is a schematic block diagram of an illustrative implantable medical device 10. the illustrative medical device 10 may be adapted to be implanted at a position at or near the hepatic neural plexus of a patient and may be used to stimulate the hepatic neural plexus of the patient. In some instances, stimulation of the hepatic neural plexus from a position proximate the hepatic neural plexus may yield quantifiable improvements in metabolic disorders such as Type 2 diabetes, even with applying stimulation for only minutes per day.

In some instances, the implantable medical device 10 may be designed to provide for MRI compatibility. In some cases, this may include limiting the amount of magnetic material in the implantable medical device 10. In some cases, this may include optimizing the geometry of conductive material within the implantable medical device 10. In some cases, the implantable medical device 10 may include features that minimize or compensate for electromagnetic induction which could otherwise impact performance of the implantable medical device 10 or cause harm to the patient.

The implantable medical device 10 includes a stimulation device 12 that is adapted to provide energy to the patient's hepatic neural plexus. A power supply 14 is adapted to supply power to the stimulation device 12. The power supply 14 may include one or more batteries. The power supply 14 may include one or more rechargeable batteries. The power supply 14 may include one or more capacitors. In some instances, the power supply 14 may include a circuit (not shown) that enables a rechargeable battery or a capacitor within the power supply 14, to be charged or recharged via an electrical field generated outside of the body, or at least outside of the implantable medical device 10. In some instances, the power supply 14 may include an energy harvesting mechanism that allows the implantable medical device 10 to capture energy from the patient's body. Such captured energy may be used to directly power the stimulation device 12 and other components of the implantable medical device 10, or to charge or recharge a battery or capacitor that is subsequently used to power the stimulation device 12 as well as other components of the implantable medical device 10.

A controller 16 is operably coupled to the stimulation device 12 and to the power supply 14. In some instances, the controller 16 may be adapted to control operation of the stimulation device 12 by modulating voltage and/or current delivery, for example, to the stimulation device 12. The controller 16 may be adapted to communicate in order to allow for adjustment of the power, pulse frequency, continuous pulse duration, pulse repetition frequency, duration of time that pulses may be repeated, or other control parameters (set desired blood sugar level, etc.). In some instances, the controller 16 may be configured to communicate to a software application on a mobile device, or with other wearable sensors or devices (e.g., smart watch, GPS enabled device, cameras, health metric monitoring and planning applications).

In some instances, the controller 16 may be adapted to communicate with other options systems in order to improve treatment. As an example, the controller 16 may be adapted to communicate with a system that is adapted to detect glucose levels or other molecular or physiological signals that may indicate effectiveness of the treatment provided by the implantable medical device 10. The controller 16 may be adapted to communicate with a system or mechanism that is adapted to monitor digestive indicators such as chemical signals or motion of digestive organs or muscles. The controller 16 may be adapted to communicate with a system that is adapted to control production or release of drugs or hormones. As an example, the controller 16 may be adapted to communicate with an artificial pancreas or an insulin pump. In some instances, the controller 16 may be adapted to communicate with a system that is adapted to compute an appropriate adjustment of energy modulation to optimize treatment based on feedback from the physiological monitoring system or other integrated systems.

In some instances, the implantable medical device 10 may further include a receiver 18 that is adapted to receive information that is transmitted to the implantable medical device 10. In some instances, for example, the receiver 18 may be adapted to receive one or more control parameters that the controller 16 may use in controlling operation of the stimulation device 12. In some instances, the receiver 18 may be adapted to receive information from a sensor 20. The sensor 20 may be implanted remotely from the implantable medical device 10, for example. In some instances, the sensor 20 may be part of the implantable medical device 10. The sensor 20 may provide information to the receiver 18 that the controller 16 may utilize in controlling operation of the stimulation device 12. As an example, the sensor 20 may be a blood glucose sensor, and may provide a signal representative of a sensed blood glucose level. Other types of sensors are also contemplated.

In some instances, the implantable medical device 10 may further include a transmitter 22 that is adapted to transmit information from the implantable medical device 10 to a remote device 24. In some instances, for example, the transmitter 22 may be adapted to transmit information from the implantable medical device 10 such as a remaining power level in the power supply 14. In some instances, the receiver 18 may receive a signal from the sensor 20 and the transmitter 22 may subsequently relay the signal to the remote device 24. In some instances, the remote device 24 may represent another implanted device that is remote from the implantable medical device 10. In some instances, the remote device 24 may represent an external device that is outside of the patient. Examples of such devices may include a monitoring station or a power station.

The receiver 18 and the transmitter 22 may communicate with the sensor 20 and the remote device 24, respectively, using a variety of different communication protocols. In some instances, the receiver 18 and the transmitter 22 may communicate with the sensor 20 and the remote device 24, respectively, using a wireless communication protocol. In some instances, the receiver 18 and the transmitter 22 may communicate with the sensor 20 and the remote device 24, respectively, using a wired communication protocol using conductive wires (not shown). In some instances, the receiver 18 and the transmitter 22 may communicate with the sensor 20 and the remote device 24, respectively, using conducted communication in which electrical signals pass through body tissue.

In some instances, the implantable medical device 10 may further include a housing 26 (shown in phantom) that is adapted to house the power supply 14 and the controller 16. In some instances, the stimulation device 12 may be outside of the housing 26, and may be coupled with the power supply 14 and the controller 16 via an electrical cable that extends between the power supply 14 and the controller 16 and the stimulation device 12. In some instances, the implantable medical device 10 may instead further include a housing 28 that is adapted to house the stimulation device 12, the power supply 14, and the controller 16. The housing 26 and 28 may be adapted to be implanted near a desired treatment site within the patient.

As an example, the housing 26 and 28 may be adapted to be implanted near the hepatic neural plexus of the patient. In some instances, the housing 26 and 28 may be adapted to be implanted within an hepatic portal vein of the patient. As another example, the housing 26 and 28 may be adapted to be implanted on or within a heptatoduodenal ligament of the patient. Other implantation sites are also contemplated.

In some instances, when the implantable medical device 10 is entirely contained in a single housing such as the housing 28, the stimulation device 12 may be located at one end of the housing 28. The housing 28 may be compact in order to be delivered and to fit within the porta hepatis without negatively impacting the functionality of adjacent anatomy such as the portal vein, proper hepatic artery, bile ducts, etc. In some instances, the implantable medical device 10 may be delivered elsewhere, but may still be appropriately sized to have minimal displacement or impact on visceral organs or adjacent anatomy. One exception could be if an increased size were to serve a beneficial purpose, such as displacing volume in or around the stomach as a method of treating obesity (similar to an intra-gastric balloon or gastric restrictive surgery).

In some instances, the housing 26 may house the power supply 14 and the controller 16, but the stimulation device 12 may be located outside of the housing 26, with a cable connecting the stimulation device 12 with the power supply 14 and the controller 16. In some instances, the housing 26 may not require implantation in the porta hepatis but could be further away in a more accessible location such as under the patient's skin in the abdomen. During implantation, the cable (not shown) and the stimulation device 12 may be routed into the porta hepatis region and placed within range of the hepatic neural plexus.

In some instances, the stimulation device 12 may be adapted to utilize electrical energy in order to stimulate the hepatic neural plexus of the patient, and thus may include a pair of electrical leads that each include an uninsulated electrically conductive portion that may function as an electrode that may be placed in contact with the hepatic neural plexus of the patient, for example. Portions of the electrical leads other than the electrodes may be covered in an electrically insulating material.

In some instances, the stimulation device 12 may include a transducer. In some instances, a transducer may not require all components to be placed in close proximity of the targeted nerves. An electromechanical transducer may have a small component implanted at the targeted nerves, such as a small electromagnet or permanent magnet that is moved by a magnetic field created by a larger coil from a further distance (possibly outside the body). This is just an example. In some instances, a transducer may include a piezoelectric material, a micro-electronic mechanical system (MEMS), an electromagnetic linear actuator, or an electromagnetic eccentric rotating mass, any means of using electrical current to move a component and impart force on the neurons, a resistive element that generates heat from an electrical current, or a light emitting element (to heat the tissue).

In some instances, magnetic neural modulation may be employed. For example, in some instances, alternating magnetic currents may be used to modulate neural activity, and doesn't require direction conduction of current. In some instances, use of alternating magnetic currents may eliminate some issues pertaining to biocompatibility and chemical erosion of electrodes.

In some instances, the transducer may be adapted to use mechanical energy in modulating the hepatic neural plexus of the patient. In cases in which the stimulation device 12 is a transducer that utilizes mechanical energy, it can be useful to note that a range of the transducer may be defined as the region in which the required pressure and intensity (power per unit area) is sufficient to activate the targeted nerves. For a single mechanical transducer this region may lie in very close proximity, perhaps within 5 millimeters or less from the transducer. A material with a different compressive velocity may be shaped in a concave or convex form to focus the energy to increase the pressure and intensity in a smaller area, or an area further from the transducer. A mechanical system may be used to rotate or translate the position of the transducer after implantation to correct for placement inaccuracy during implantation. Multiple transducers may be used to either provide redundancy in the placement and increase the odds that one of the various transducers are in close enough proximity to the nerves to deliver adequate energy, or to form an array capable of beamforming. A beamforming array would allow the energy to be focused and targeted at different angles and distances from the transducer, allowing more adjustment in the position of energy delivery after implantation and the ability to implant the device further away from the hepatic neural plexus. A mechanical transducer placed out of immediate proximity to the hepatic neural plexus may need to compensate for the effects of differences in mechanical or acoustic impedance (ratio of pressure to particle velocity) or compressive velocity across different tissues, liquids, or gases.

In some instances, the transducer may be adapted to use electrical energy in modulating the hepatic neural plexus of the patient. In some instances, the transducer may be adapted to use thermal energy in modulating the hepatic neural plexus of the patient. In some instances, the transducer may be adapted to use ultrasonic energy in modulating the hepatic neural plexus of the patient. Ultrasonic energy may be provided by an ultrasound radiating member. As used herein, the term “ultrasound radiating member” refers to any apparatus capable of producing ultrasonic energy. For example, in one embodiment, an ultrasound radiating member may include an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz or lead zirconate titanate. While lead zirconate titanate is used in many medical ultrasound transducers, in some cases quartz may be more suitable for an implantable device in terms of biocompatibility. In some instances, piezoelectric ceramics may change shape when an electrical voltage is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

The implantable medical device 10 may be implanted via any of a variety of different procedures. For example, the implantable medical device 10 may be implanted using open surgery, laparoscopic surgery, endoscopic surgery, catheter-based surgery, or a combination of surgical access methods. Open surgery may be considered as more invasive and thus less ideal, although there may be circumstances for particular patients in which open surgery is a satisfactory option.

During a laparoscopic procedure, the implantable medical device 10 may be placed directly through a small incision in the skin. The porta hepatis may be accessed from below the liver. The implantable medical device 10 may be implanted with the stimulation device 12 disposed adjacent to the hepatic neural plexus. If the stimulation device 12 is separate from the housing 26 including the power supply 14 and the controller 16, the housing 26 including the power supply 14 and the controller 16 may be implanted below the skin, with a cable running between the housing 26 including the power supply 14 and the controller 16 and the stimulation device 12.

During an endoscopic procedure, the porta hepatis may be accessed by cutting through the stomach wall or the duodenal wall, or by entering either the bile duct or the cystic duct. In some cases, any portion of the implantable medical device 10 implanted within the bile duct or the cystic duct may include an internal lumen that allows for fluid flow through the implantable medical device 10 in order to not block fluid flow through the bile duct or the cystic duct. The stimulation device 12 may be placed within the porta hepatis, either with or without the rest of the implantable medical device 10. In some instances, the housing 26 including the power supply 14 and the controller 16 may be placed in the stomach, the duodenum or in interstitial space between organs or under the skin. In instances in which the stimulation device 12 is an array of transducers or a focusing element, the increased range may allow the entirety of the implantable medical device 10 to be placed in the stomach, the duodenum or in interstitial space between organs or under the skin.

During a catheter-based surgery, one or more catheters may be introduced through the skin into a large vein or artery and then navigated to the portal veil or the hepatic artery, both of which are adjacent to the hepatic neural plexus. The stimulation device 12 may be delivered within the vascular lumen and may be actively secured with anchors, barbs, tines or sutures, or may be passively secured by including a biasing force to bias towards the vessel wall. In some instances, the vascular wall may be punctured in order to deliver the stimulation device 12 into the porta hepatis. In some instances, this may include having to seal the puncture to prevent hemorrhage.

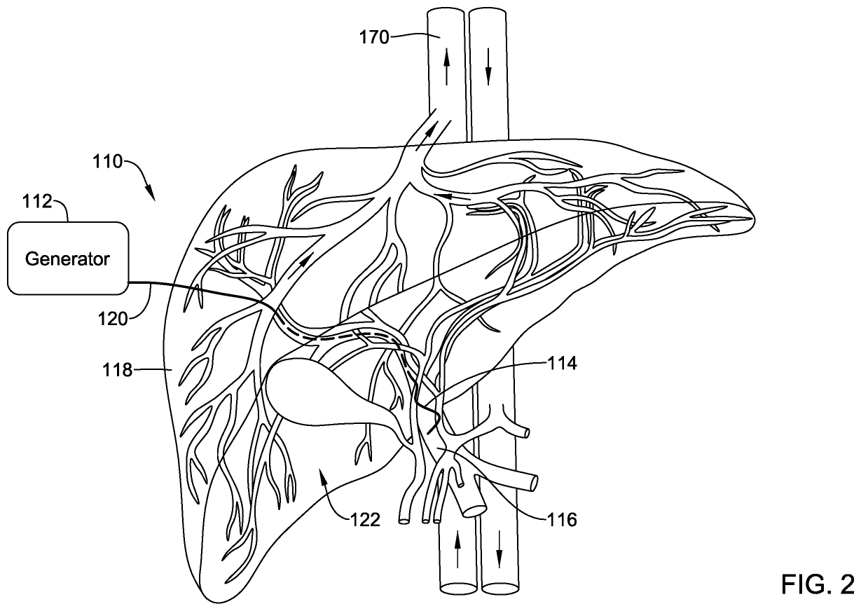

FIG. 2 is a schematic illustration of a portion of a person's anatomy, showing implantation of an implantable medical device 110 in an intraluminal position. The implantable medical device 110 includes a generator portion 112 and a stimulation portion 114. The generator portion 112 may be considered as being an example of the housing 26, the power source 14 and the controller 16. The stimulation portion 114 may be considered as being an example of the stimulation device 12. The generator portion 112 may be seen as being implanted near but not adjacent the stimulation portion 114. The stimulation portion 114 is implanted within a patient's hepatic portal vein 116 while the generator portion 112 is disposed near the patient's liver 118. A lead 120 extends between the generator portion 112 and the stimulation portion 114. The lead 120 includes one or more electrical conductors that carry energy from the generator portion 112 to the stimulation portion 114.

In some instances, a transhepatic access procedure may be used to implant the stimulation portion 114, targeting the portal vein instead of the hepatic vein. In some cases, injecting contrast into the splenic vein, the inferior mesenteric vein or the superior mesenteric vein may be used to provide optimal viewing for placement. In some instances, abdominal ultrasound may be used instead of fluoroscopy for guidance. In some instances, the common bile duct 122 may be targeted. In some cases, this may require a combination of ERCP (endoscopic retrograde cholangiopancreatography) to deliver the stimulation portion 114 and the lead 120, and open/endoscopic/laproscopic surgery to deliver the generator portion 112. In some instances, the generator portion 112 may be placed in the interstitial subhepatic space, or possibly fixed within the GI (gastrointestinal) tract.

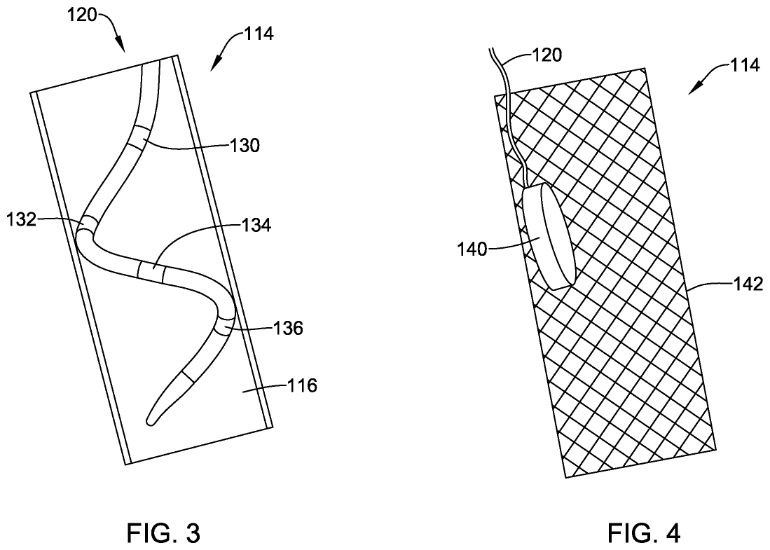

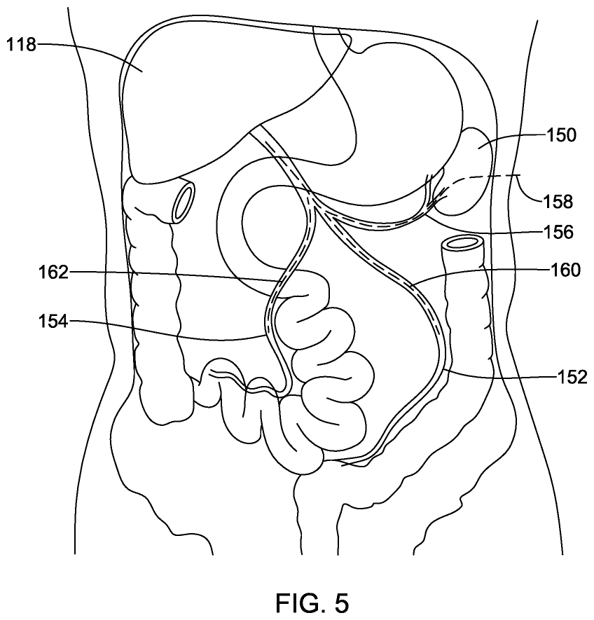

For intraluminal placement, the stimulation portion 114 may take a variety of forms. For electrical or ultrasonic stimulation, the stimulation portion 114 may take the form of a helix-shaped lead or probe, as seen in FIG. 3. In some instances, the helix may have a dimension that allows a radially exerted spring force on the vessel wall that holds the helix in place. For mechanical or ultrasonic stimulation, the stimulation portion 114 may take the form of a mechanical transducer such as a vibration motor or an actuator, or an ultrasound transducer. The ultrasonic transducer may be piezoelectric or MEMS (Micro-Electro Mechanical System). There may be a single ultrasonic transducer or an array of ultrasonic transducers for redundancy, selectivity and possible beam-steering. The transducer may be affixed to a stent and held in position within the hepatic portal vein 116 via the stent. This can be seen in FIG. 4. In some instances, additional anchoring methods may be utilized to prevent migration. In some instances, the generator portion 112 may be placed subcutaneously, or in a submuscular abdominal pocket, or in other suitable positions in the abdomen. If the generator portion 114 include wireless charging capabilities, the depth of the generator portion 114 from the skin should be considered for charging capabilities.

FIG. 3 shows the lead 120 having a corkscrew shape, disposed within the hepatic portal vein 116. The lead 120 may include one or more treatment elements 130, 132, 134 and 136 arranged axially along a length of the lead 120. While a total of four treatment elements 130, 132, 134 and 136 are shown, it will be appreciated that this is merely illustrative, as the lead 120 may include any number of treatment elements. In some instances, the treatment elements 130, 132, 134 and 136 may represent electrodes that may be used for electrical stimulation, with the generator portion 112 controlling the electrical pulses provided to the treatment elements 130, 132, 134 and 136. In some instances, the treatment elements 130, 132, 134 and 136 may each represent ultrasound transducers, with the generator portion 112 controlling operation of the ultrasound transducers.

FIG. 4 shows the lead 120 in a linear configuration, extending to and coupled with a transducer 140 that is mounted to a stent-like structure 142. In some instances, the transducer 140 may be configured to provide mechanical stimulation to the hepatic neural plexus. As an example, the transducer 140 may represent a mechanical transducer, or perhaps two or more mechanical transducers. In some cases, the transducer 140 may generate physical movement when stimulated or actuated. In some cases, mechanical compression and rarefaction of neurons may activate mechanosensitive ion channels, and thus can provide stimulation to the hepatic neural plexus. A mechanical transducer may include a vibration motor or an actuator, or an ultrasound transducer. The ultrasonic transducer may be piezoelectric or MEMS (Micro-Electro Mechanical System), and may be stimulated by a source of electricity.

In some cases, the stent-like structure 142 may be moveable between a collapsed configuration for delivery and an expanded configuration (as shown) for deployment. The stent-like structure 142 may be adapted to have a biased configuration in which the stent-like structure 142 has an overall diameter that is sufficient to engage the vessel walls of the hepatic portal vein 116 (not shown in FIG. 4).

In some instances, delivery of the implantable medical device 110 may involve delivery via the spleen or via the mesenteric veins. FIG. 5 is a schematic view of a portion of the anatomy, showing, among other components, the liver 118, a spleen 150, an inferior mesenteric vein 152 and a superior mesenteric vein 154, and a splenic vein 156. As can be seen, each of the inferior mesenteric vein 152, the superior mesenteric vein 154 and the splenic vein 156 provide a route to a location proximate the hepatic neural plexus.

A route 158 may be seen as starting at the patient's skin near the spleen 150. Penetrating the skin to reach the spleen 150 allows access via the spleen 150 to the splenic vein 156. A route 160 follows the inferior mesenteric vein 152 and a route 162 follows the superior mesenteric vein 154. In some instances, when placing the probe/lead in the portal vein, delivering the implantable medical device 110 upstream would allow contrast to be injected and flow downstream to visualize the portal vein under fluoroscopy. Mesenteric vein (superior or inferior) access may require ultrasound guidance to avoid perforating the GI tract or other organs. The generator 112 may be placed subcutaneously or in a submuscular abdominal pocket.

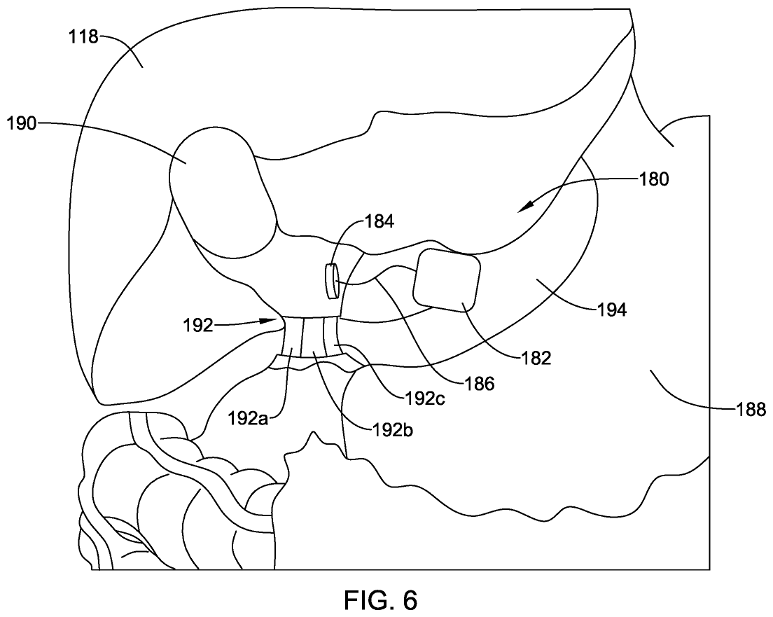

FIG. 6 is a schematic view of a portion of a patient's anatomy, representing an interstitial or subhepatic placement of an implantable medical device 180. The implantable medical device 180 includes a generator portion 182 and a stimulation portion 184. The generator portion 182 may be considered as being an 184 of the housing 26, the power source 14 and the controller 16. The stimulation portion 184 may be considered as being an example of the stimulation device 12. The generator portion 182 may be seen as being implanted near but not adjacent the stimulation portion 184. A lead 186 extends between the generator portion 182 and the stimulation portion 184.

FIG. 6 shows anatomy in addition to the liver 118, including a stomach 188, a gall bladder 190 and a hepatoduodenal ligament 192. The heptatoduodenal ligament 192 includes the common bile duct 192a, the portal vein 192b and the proper hepatic artery 192c. The stimulation portion 184 may be placed on or within the hepatoduodenal ligament 192 in proximity to the hepatoportal nerve plexus. The stimulation portion 184 should be fixed to avoid migration, and may for example be fixed to the hepatoduodenal ligament 192 or other connective tissue. In some instances, the generator portion 182 may be fixed to the same tissue or elsewhere, such as the lesser ormentum 194 or more superficial periotoneum. If delivered laparoscopically, the generator portion 182 may be fixed to the ribs. It will be appreciated that for wireless charging, depth of placement may be important.

In some instances, if the implantable medical device 180 is small enough, the implantable medical device 180 may be delivered laparoscopically or endoscopically by placing the stimulation device 184 at the porta hepatis and attaching the implantable medical device 180 to the hepatoduodenal ligament 192. In some instances, the implantable medical device 180 may be attached using clips, tines or active fixation screws. As an example, the tines or screws may pierce the connective tissue. When the generator portion 182 is a separate piece, it may be placed and fixed. If delivered endoscopically, fluoroscopy and/or abdominal ultrasound may be used to assist guidance.

In some instances, accurate placement is important for effectively modulating metabolic responses. Placement during delivery may be monitored or confirmed with external abdominal ultrasound, endoluminal ultrasound (radial ultrasound with a rotational element or radial phased array, or planar ultrasound with a phased array such as those commonly used in ICE (intracardiac echocardiography) catheters. Ultrasound information may be acquired by the implant itself. The implant may use basic doppler ultrasound to determine proximity to the vein or artery, or B-mode imaging, M-mode imaging, color doppler imaging, or other imaging modes. In some instances, transducer design may be different for imaging and stimulation delivery than just stimulation delivery alone; imaging requires higher acoustic bandwidth and shorter pulse lengths. Additional electronic circuitry may be required to receive the ultrasound signals in addition to transmitting them. Data transmission for displaying the image utilize wired or wireless protocols.

In some instances, placement may be monitored or confirmed by measuring neurological electrical signals to determine when the lead/transducer is in proximity to the hepatoportal plexus. The signals could be identified as any electrical signal or neurological origin, or specific signal patterns associated with the hepatoportal plexus. Fluoroscopy/3D fluoro reconstructions/cone beam CT (all require fluoroscopic contrast, might be difficult to deliver contrast to reveal the hepatic portal vein. Could be injected into the aorta somewhere to reveal the hepatic artery but would need lots of contrast may also be used.

After implantation, devices with multiple electrical leads or transducers may allow the operator to modulate the energy delivery on the individual leads while monitoring biomarkers or physiological signals such as blood glucose levels, in order to optimize the energy delivery towards specific areas. Devices with multi-element ultrasound transducers may use known beamforming techniques to focus and steer the energy by phasing the activation of individual elements. The focal point may be calculated from spatial information derived from imaging methods or other ultrasound measurements mentioned above, taken during or after implantation. Imaging or otherwise measuring data with the implanted transducer may offer the added benefit of being able to monitor the migration of the device over the lifetime of the device. If the ultrasound signal or image changes, then it may indicate migration. Devices integrated with closed loop feedback from a physiological monitoring system (e.g., a blood glucose monitor) may use that feedback to automatically adjust and optimize the energy delivered by individual leads (which leads are on/off, amount of power, pulse sequence, etc.) or where the energy is steered and focused by an ultrasound array.

In some instances, delivery may involve passage through the inferior vena cava 170 (FIG. 2). The implantable medical device 10, 110, 180 may be delivered through a subclavian or jugular vein, or other peripheral vein in the torso that feeds into the inferior vena cava 170. The implantable medical device 10, 110, 180 may be inserted down the inferior vena cava 170, into the hepatic vein and extraluminally cross to the portal vein for implantation.

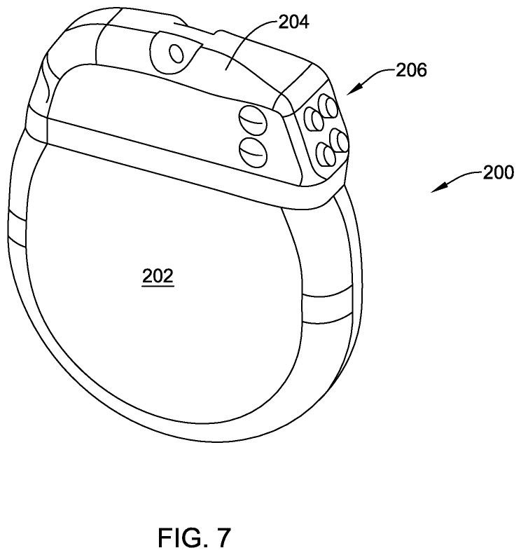

FIG. 7 is a schematic view of an illustrative housing 200 that may be considered as being an example of the housing 26 (including the power supply 14 and the controller 16), or perhaps the housing 28 (also including the stimulation device 12). In some instances, the housing 200 includes a main body 202 and a header 204. In some cases, the header 204 includes one or more connections 206 for connecting additional leads that may be used to connect the header 204 to a sensor, for example, or to the stimulation device 12 when the stimulation device 12 is remote from the rest of the implantable medical device 10, 110, 180.

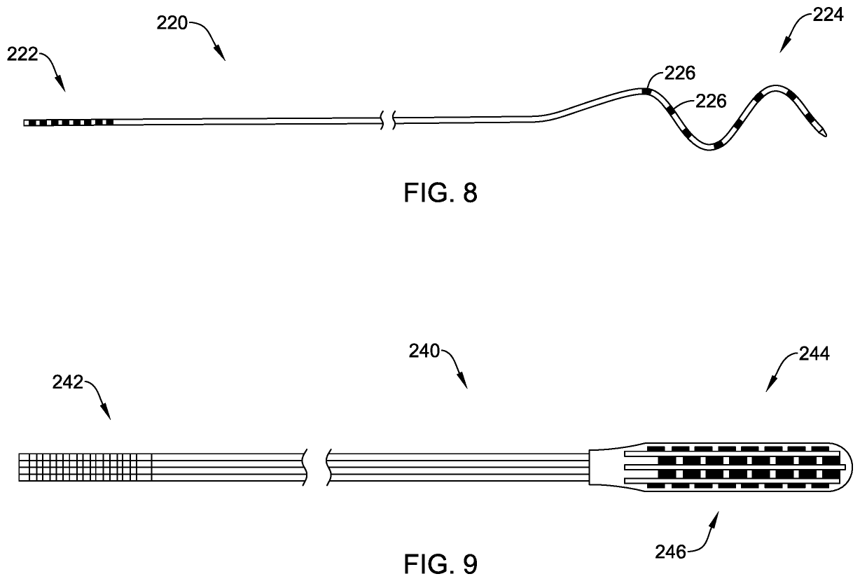

FIG. 8 is a schematic view of an illustrative intraluminal lead 220 extending from a proximal region 222 to a distal region 224. The intraluminal lead 220 may be considered as being an example of the lead 120 or 186. The intraluminal lead 220 may have a length that varies from 200 millimeters to 1000 millimeters (1 meter), or longer, depending on the spacing between access point and generator placement are far apart. The intraluminal lead 220 includes a spiral configuration within the distal region 224. In some instances, the spiral configuration has a length of 10 to 150 millimeters, and may have a spiral diameter that is in a range of 5 to 20 millimeters. A total of eight electrodes 226 are shown within the spiral configuration, although in some instances the spiral configuration may include fewer than eight electrodes 226, or may even include nine or more electrodes 226.

FIG. 9 is a schematic view of an illustrative interstitial lead 240 extending from a proximal region 242 to a distal region 244. The interstitial lead 240 may have a length of 100 to 500 millimeters, for example. In some instances, the distal region 244 includes an array 246 of electrodes. In some instances, the array 246 may include a large number of electrodes. As an example, the array 246 may have a length of 40 millimeters, a diameter of 10 millimeters and may include a total of 32 electrodes.

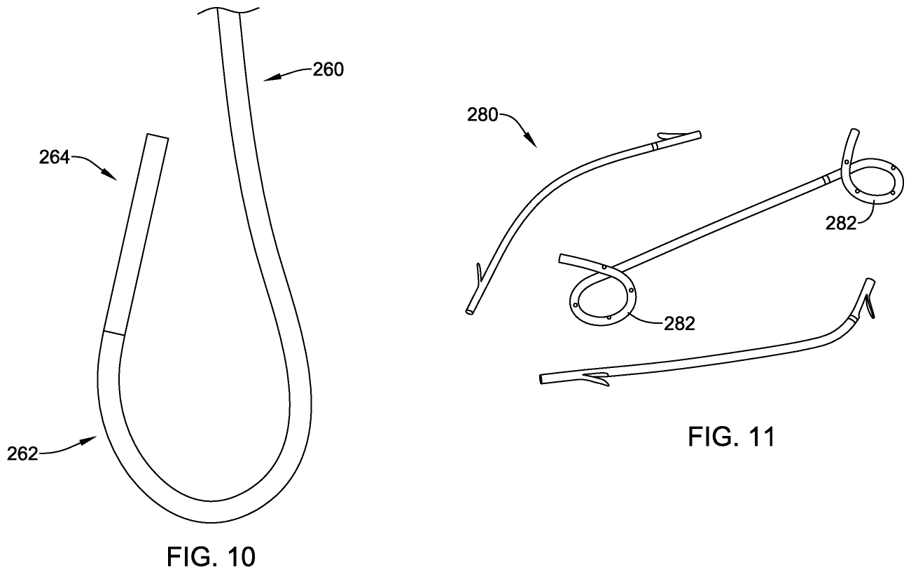

FIG. 10 is a schematic view of an illustrative lead 260 that includes a J-shaped distal region 262. In some instances, the J-shaped distal region 262 may be beneficial in wedging the J-shaped distal region 262 into a lumen in order to secure. In some cases, the lead 260 (and other leads such as the intraluminal lead 220 and the interstitial lead 240) may include silicone tines for securement within smaller vessel branches for additional holding power. The J-shaped distal region 262 may include a stimulation region 264.

FIG. 11 is a schematic view of several illustrative tube-like stents 280. The tube-like stents 280 may include pigtails or flaps 282 to help reduce migration. The pigtails or flaps 282 may be made of plastics or flexible thin-walled metals. Metals may be laser cut to increase flexibility. Especially useful for biliary placement. Transducers or electrodes may be placed throughout the stent or constrained to one area that is intended to be placed proximal to the hepatoportal nerve plexus. For biliary placement, electrodes/transducers may only be placed on one end that is inserted up to the perihilar region of the common bile duct. Any GI/bile duct placement may require additional considerations to prevent contamination from devices crossing between the ducts or GI tract and the sterile interstitial space.

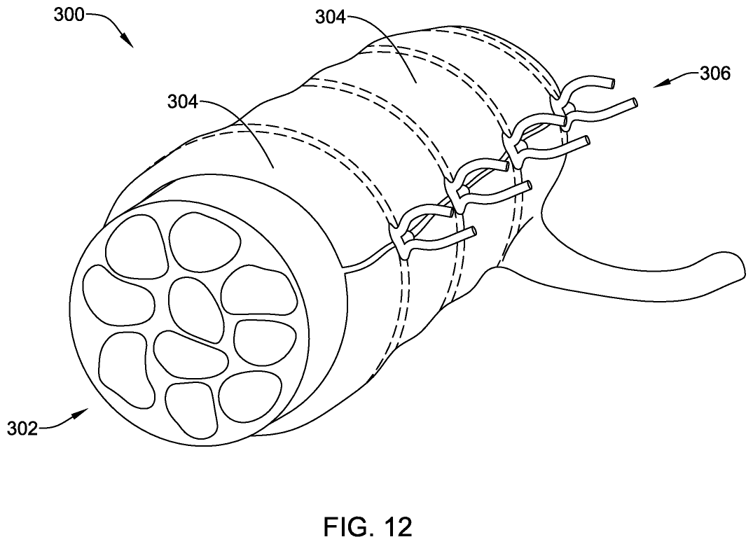

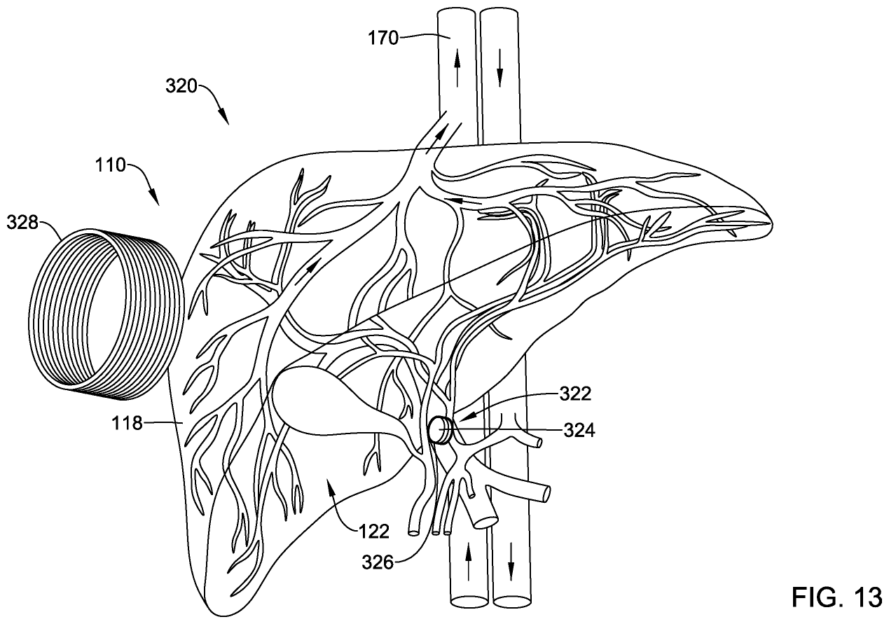

FIG. 12 is a schematic view of an illustrative lead 300 having a cuff design. The lead 300 is shown disposed about a vagus nerve 302, but this is just an example. The cuff design may wrap around the entire hepatoduodenal ligament or (if inserted through the connective tissue) the common hepatic artery, portal vein and intertwined neural plexus. This design may require laparoscopic or open surgical placement. The cuff design includes a number of individual cuffs 304 that may be held together via a number of sutures 306.

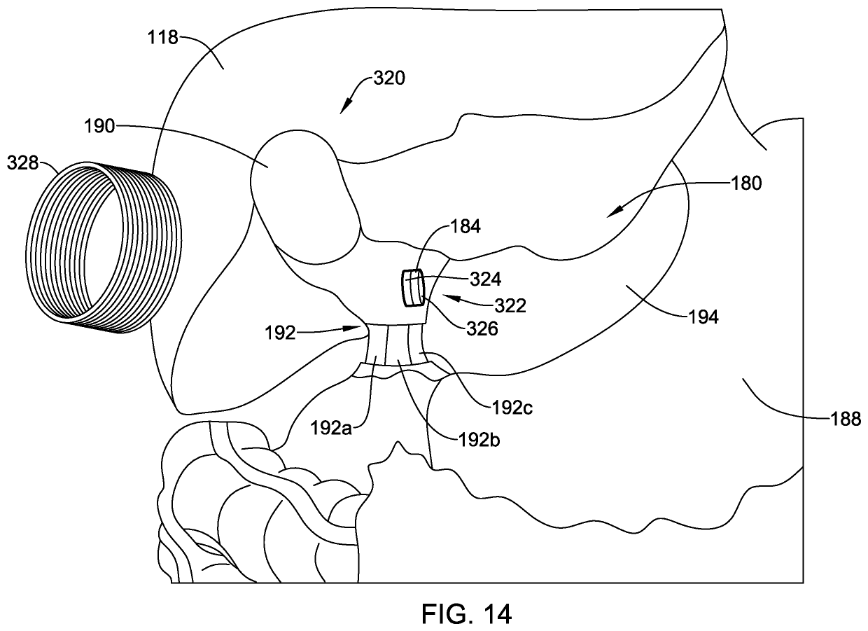

FIG. 13 is a schematic view of a portion of a patient's anatomy, illustrating a possible intravascular implantation of a system 320 for stimulating the hepatic neural plexus. FIG. 14 is a schematic view of a portion of a patient's anatomy, illustrating a possible intravascular implantation of the system 320 for stimulating the hepatic neural plexus. The system 320 includes a stimulation device 322 that is adapted for implantation near the patient's hepatic neural plexus and is adapted to provide mechanical energy to the patient's hepatic neural plexus. In some cases, the stimulation device 322 may include a mechanical transducer, such as that discussed with respect to the transducer 140 in FIG. 4. In some cases, the stimulation device 322 may include a magnet 324. As an example, the magnet 324 may be a permanent magnet. As another example, the magnet 324 may be hermetically encapsulated within a housing 326. In some cases, the magnet 324 may be a neodymium magnet. The magnet 324 may be considered as having a North pole and a South pole, and thus will oscillate or otherwise move back and forth in response to an applied alternating magnetic field. The system 320 includes a source of excitation energy 328. In some cases, the source of excitation energy 328 may be a magnetically inductive coil that may be placed against a patient's skin and an alternating current may be applied to the source of excitation energy 328 in order to cause the magnet 324 to oscillate. In some cases, the source of excitation energy 328 may be placed over the patient's abdomen. For obese patients, it may work better to place the source of excitation energy 328 against the patient's side or even the patient's back. In some cases, operation of the source of excitation energy 328 may be controlled by the controller 16 (FIG. 1).

In some cases, the housing 326 may be adapted to be laparoscopically implanted. In some instances, the housing 326 may be adapted to be intravascularly implanted. In some instances, the housing 326 may be adapted to be implanted near the hepatic neural plexus of the patient. In some case, the housing 326 may be adapted to be implanted within an hepatic portal vein of the patient. In some cases, the housing 326 may be adapted to be implanted on or within a heptatoduodenal ligament of the patient.

In some instances, particularly when the magnet 324 is implanted intravascularly, it may be helpful to secure the magnet 324 to a stent-like structure 142 such as that shown in FIG. 4 in order to fix the magnet 326 in place within the vasculature. In some cases, the magnet 324 may be placed against the wall of the vessel in order to maximize vibrations being transmitted to the neural plexus. In some cases, low frequency compressive waves do not transmit well through blood.

In some cases, the controller 16 (FIG. 1) may be adapted to provide an alternating current to the inductive coil in order to generate an alternating magnetic field that provides the excitation energy to the stimulation device. As an example, the controller 16 may be adapted to provide an alternating current to the inductive coil at an AC frequency ranging from 30 Hertz (Hz) to 20,000 Hz.

In some instances, the aspect ratio of the source of excitation energy 328 may impact the directivity and focus of the magnetic field. A more directive focus will allow higher flux density to transmit deeper into the patient to reach the magnet. Several factors may be considered for optimal placement of the magnet 324 during implantation. For example, the axis of magnetic vibration should be aligned with the hepatic neural plexus to optimize the mechanical compression and rarefaction of the neurons to activate the mechanosensitive ion channels. The axis of magnetic vibration will depend on the axis of the magnetic dipole. In some cases, users with thicker layers of abdominal body fat may need to place the source of excitation energy 328 more laterally on their side, or posteriorly on their back to minimize the distance between the source of excitation energy 328 and the magnet 324. The dipole axis of the magnet 324 should be aligned with the axis of the source of excitation energy 328 to maximize the efficiency of generating mechanical force and displacement.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-clastic nitinol in that the linear elastic and/or non-super-elastic nitinol does not display a substantial “superelastic plateau” or “flag region” in its stress/strain curve like super-clastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear elastic and/or non-super-elastic nickel-titanium alloy maintains its linear elastic and/or non-super-clastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system adapted to modulate an hepatic neural plexus of a patient, the system comprising:
a stimulation device adapted for implantation near the patient's hepatic neural plexus and to provide mechanical energy to the patient's hepatic neural plexus;
a source of excitation energy adapted to provide excitation energy to the stimulation device; and
a controller operably coupled to the source of excitation energy and adapted to control the source of excitation energy.

2. The system of claim 1, wherein the stimulation device is adapted to provide mechanical energy to the patient's hepatic neural plexus by undergoing physical movement.

3. The system of claim 2, wherein:
the stimulation device comprises a permanent magnet; and
the source of excitation energy comprises an inductive coil placed against the patient's skin in order to provide excitation energy to the permanent magnet.

4. The system of claim 3, wherein the permanent magnet comprises a neodymium magnet.

5. The system of claim 3, wherein the permanent magnet comprises a hermetically encapsulated permanent magnet.

6. The system of claim 5, wherein the permanent magnet is hermetically encapsulated within a housing.

7. The system of claim 6, wherein the housing is adapted to be laparoscopically implanted.

8. The system of claim 6, wherein the housing is adapted to be intravascularly implanted.

9. The system of claim 6, wherein the housing is adapted to be implanted near the hepatic neural plexus of the patient.

10. The system of claim 6, wherein the housing is adapted to be implanted within an hepatic portal vein of the patient.

11. The system of claim 6, wherein the housing is adapted to be implanted on or within a heptatoduodenal ligament of the patient.

12. The system of claim 3, wherein the controller is adapted to provide an alternating current to the inductive coil in order to generate an alternating magnetic field that provides the excitation energy to the stimulation device.

13. The system of claim 12, wherein the controller is adapted to provide an alternating current to the inductive coil at an AC frequency ranging from 30 Hertz (Hz) to 20,000 Hz.

14. The system of claim 2, wherein the stimulation device comprises a piezoelectric material and the source of excitation energy comprises a source of electricity.

15. The system of claim 2, wherein the stimulation device comprises an eccentric rotating mass.

16. A system adapted to modulate an hepatic neural plexus of a patient, the system comprising:
a magnetic stimulation device adapted for implantation near the patient's hepatic neural plexus and to provide mechanical energy to the patient's hepatic neural plexus; and
an external magnetic field generator adapted to excite the magnetic stimulation device and cause movement of the magnetic stimulation device.

17. The system of claim 16, wherein the magnetic stimulation device comprises a neodymium magnet.

18. The system of claim 16, wherein the magnetic stimulation device comprises a hermetically encapsulated permanent magnet.

19. The system of claim 16, wherein the external magnetic field generator is adapted to generate an alternating magnetic field in response to an applied AC current.

20. A system adapted to modulate an hepatic neural plexus of a patient, the system comprising:
an encapsulated permanent magnet adapted for implantation near the patient's hepatic neural plexus and to provide mechanical energy to the patient's hepatic neural plexus; and
an external magnetic field generator adapted to generate an alternating magnetic field;
wherein the encapsulated permanent magnet oscillates position in response to the alternating magnetic field.
